(19) United States
(12) Patent Application Publication
(10) Pub. No.: US 2021/0212600 A1
(43) Pub. Date: Jul. 15, 2021

(54) Systems, Methods, and Devices for Evaluating Entertainment Metrics of Virtual Reality Based Content

(71) Applicant: PLAYING FORWARD LLC, Santa Monica, CA (US)

(72) Inventors: John Attard, Santa Monica, CA (US); Victor Talbot, Santa Monica, CA (US)

(21) Appl. No.: 17/141,039

(22) Filed: Jan. 4, 2021

Related U.S. Application Data
(60) Provisional application No. 62/959,373, filed on Jan. 10, 2020.

(51) Int. Cl. A61B 5/11; G16H 20/30; G16H 20/70; G06T 19/00; A63F 13/67; A63F 13/79; A63F 13/80

(52) U.S. Cl. CPC A61B 5/1124; G16H 20/30; G16H 20/70; A63F 13/80; A63F 13/67; A63F 13/79; G06T 19/003

(57) ABSTRACT

Systems and methods for evaluating the efficacy of entertaining elements during physical and cognitive therapeutic sessions are disclosed. The system is comprised of a means for providing visual and auditory information, prompting a user to perform physical and cognitive tasks of varying difficulty. The system is further comprised of a means of tracking the user's movements to provide feedback to the user and assess the performance of the user's movements. Prompted movements are designed to provide occupational therapeutic benefits to the user and may increase or decrease in difficulty based on prior assessments of a user's movements. Entertaining elements are introduced in the visual and auditory information and assessed for their impact on the user's performance of the movements.

### FIELD

The present disclosure generally relates to Virtual Reality (“VR”) devices, and more specifically to a comprehensive framework for evaluating entertainment metrics of VR-based content.

### BACKGROUND

Interactive devices such as mobile phones, tablets, smart watches, VR headsets, and the like have become a mainstay in daily life due, in part, to advances in consumer technology, increased processing power, decreased costs, improved displays and control interfaces, and so on. The readily accessible nature of such interactive devices, in turn, provides new opportunities for interfacing and interacting with users. For example, some interactive devices such as VR headsets now include interfaces (e.g., visual displays, speakers, haptic feedback hardware, etc.) for presenting content to the user, as well as new sensors (e.g., gyroscopes, accelerometers, cameras, photo detectors, microphones, etc.) for measuring physiological and/or biological activity of a user. While these interfaces and sensors provide new immersive experiences, certain challenges arise in the context of evaluating and optimizing the content presented to a user, particularly with respect to medical and/or clinical applications.

### SUMMARY

The invention disclosed is a system for providing therapeutic benefits to users with cognitive and/or physical challenges. The system consists of a means for providing visual and auditory stimuli, prompting physical movements and the performance of various cognitive tasks. The system further consists of a means of recording and measuring the movements of the user, to ascertain the user's symmetry, skill, and coordination. Measurements of the user's movements are reproduced and visually presented to the user in real-time.

The invention provides a method for prompting the user to perform physical movements of varying difficulty. Based on the user's success in performing the movements, more or less difficult successive movements are prompted.

The invention provides a method for prompting the user to perform cognitive tasks of varying difficulty. Based on the user's success in performing the tasks, more or less difficult successive tasks are prompted.

Environmental elements, such as visual and auditory stimuli, are manipulated to increase the entertainment value for the user. In addition to generic aesthetic adjustments, the invention also introduces familiar elements from popular culture, such as music and characters. The increase in entertainment value has a causative impact on the user's performance in the physical movements and cognitive tasks. The impact of the therapeutic value is measured as a Coefficient of Entertainment (“CoE”).

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 illustrates a system diagram showing a Virtual Reality (“VR”) system configured to evaluate and optimize content presented to a user, according to one example of this disclosure;

FIG. 2 illustrates a schematic block diagram of an exemplary device, such as a VR device, for evaluating and optimizing content presented to a user, according to one or more examples of this disclosure;

FIG. 3 illustrates a flow diagram showing a high-level Coefficient of Entertainment process;

FIG. 4 illustrates a flow diagram showing a high-level filter logic process; and

FIG. 5 illustrates a flow diagram showing an internal weighting adjustment process. An element or functionally similar component is indicated with the same reference number.

### DETAILED DESCRIPTION

As discussed in greater detail herein, the present disclosure is directed to a comprehensive framework for evaluating the intrinsic therapeutic effect of entertaining content presented to a user, which is represented as a Coefficient of Entertainment (“CoE”) metric. This comprehensive framework includes a feedback loop of consistent movement and cognitive measures for attributing certain characteristics to a specific user. The comprehensive framework further evaluates the content in terms of a high level/overall experience as well as the impact of more granular or specific elements of the VR experience (e.g., visual input, proximity in a virtual environment, soundscape, etc.)

Each user is unique, but patterns will emerge in which specific entertainment types will show greater impact on specific conditions or groups acting as an early stage launch consideration for maximum effect.

In order to maximize effectiveness, the disclosed system moves from correlation to causation. The approach for achieving this is to have entertaining experiences which engage the user on both the physical and cognitive level. Once in place a loop is definable with a vector focused on specific cognitive and physical objectives. The resultant CoE represents the value of this vector and is the result of the best balance between the capability of the user and the physical and cognitive challenges presented through entertaining gameplay. The more accurate the value of the CoE the greater the efficacy of each VR session and the greater the outcomes (e.g., increased mobility, improved cognitive responses, etc.)

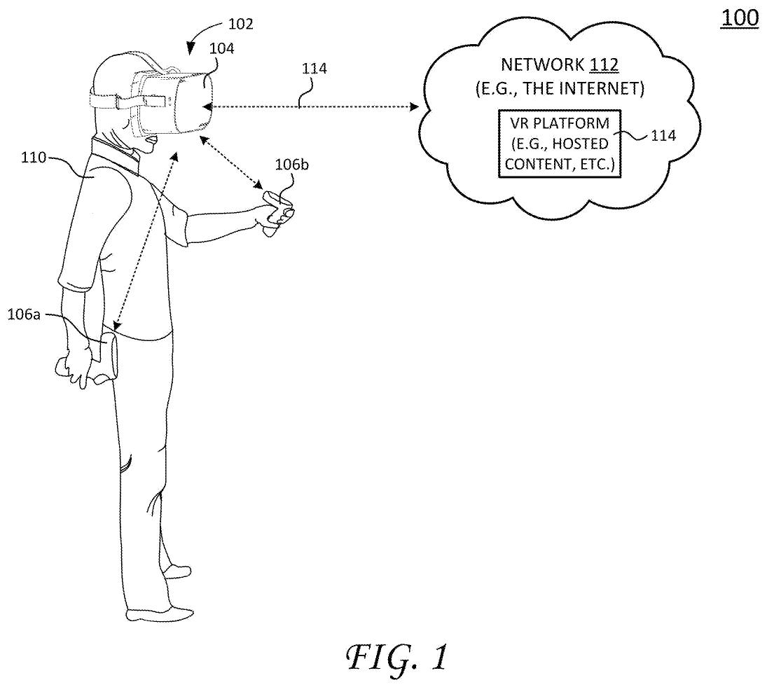

Referring to the drawings, FIG. 1 illustrates a system diagram 100, showing a VR system generally referenced as VR system 102. VR system 102 includes a wearable headset 104 worn by a patient or user 110, as well as one or more handheld controllers 106a/106b, which manipulate or interact with VR objects presented by headset 104. The components of VR system 102 communicate with each other as well as with a network 112 over communication channels or links 114.

Alternative embodiments include optional interfaces to the VR headset. A patient or user may utilize a video screen, such as a television, computer monitor, or projection screen as the source of visual input.

A communication network is a geographically distributed collection of nodes interconnected by communication links and segments for transporting data between end nodes, such as mobile devices, computers, personal computing devices (and so on), and other devices, such as network entities, sensors, etc. Many types of networks are available, ranging from local area networks (“LANs”) to wide area networks (“WANs”). LANs typically connect these nodes over dedicated private communications links located in the same general physical location, such as a building or campus. WANs, on the other hand, typically connect geographically dispersed nodes over long-distance communications links, such as common carrier telephone lines, optical light paths, synchronous optical networks (“SONET”), synchronous digital hierarchy (“SDH”) links, etc.

As shown, VR platform 114 communicates and exchanges data (e.g., traffic and/or messages) with VR system 102 using predefined network communication protocols such as certain known wired protocols (e.g., Interior Gateway Protocol (“IGP”), Exterior Border Gateway Protocol (“E-BGP”), TCP/IP), wireless protocols (e.g., IEEE Std. 802.15.4, WiFi, Bluetooth®), PLC protocols, or other shared-media protocols where appropriate. In this context, a protocol consists of a set of rules defining how the nodes interact with each other. The particular data exchanged between VR platform 114 and VR system 102 can include, for example, the illustrated hosted entertaining content as well as measured data corresponding to reactions and movements by user 110.

Those skilled in the art of computer engineering will understand that any number of nodes, devices, communication links, and the like may be used, and that the view shown herein is for simplicity. Those skilled in the art will also appreciate that the particular components of VR system 102 are illustrated for purposes of example and discussion, and further the processes for evaluating the intrinsic therapeutic effect of content presented to a user, determining the therapeutic CoE metric for such content, and optimizing the content based on the same may be configured for implementation by various types of VR systems.

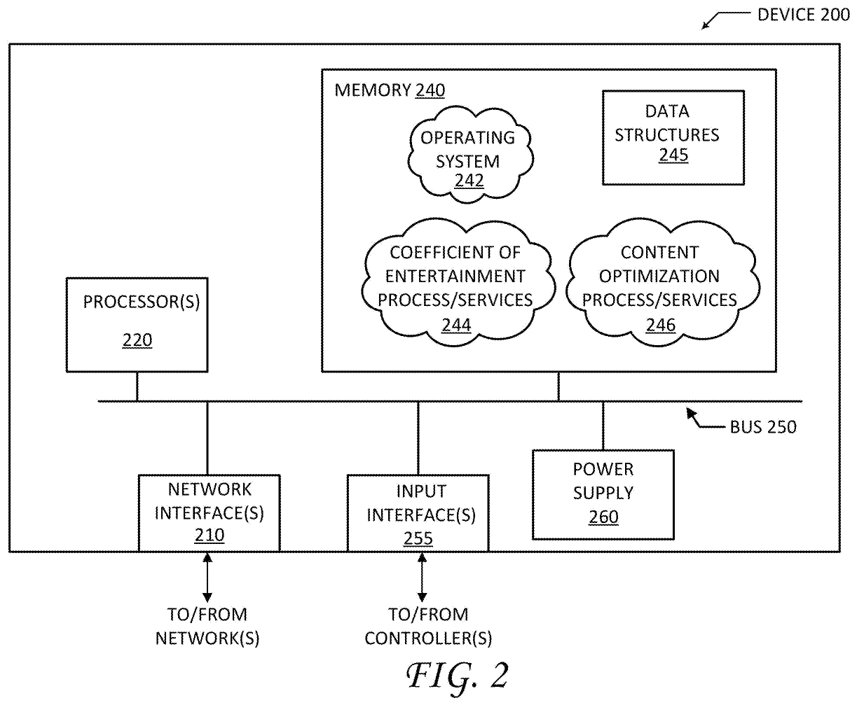

FIG. 2 illustrates a schematic block diagram of an exemplary device 200 that may be used to employ the disclosed comprehensive framework for determining a CoE metric and/or optimizing content presented to a user. In this fashion, device 200 can represent one or more components of VR system 102, or in some instances, components of VR platform 114 shown in FIG. 1 (e.g., a cloud-based or network environment).

Alternatively, it is also appreciated that one or more components of device 200 may be incorporated into a larger distributed computing environment. For example, in a distributed computing environment, the individual components of device 200 may represent logical or virtual components, where such components are implemented and hosted by a data center (e.g., using servers, distributed memory, communication links/networks, software modules, services, objects, distributed data structures, and so on), as is appreciated by those skilled in the art. However, for purposes of discussion herein, reference is made to a VR system example, where device 200 represents a component of VR system 102 (e.g., the wearable headset component).

As shown, the device 200 comprises one or more network interfaces 210, at least one processor 220, a memory 240 interconnected by a system bus 250, input interfaces 255, and a power supply 260 (e.g., battery, plug-in).

Network interface(s) 210 contain the mechanical, electrical, and signaling circuitry for communicating data over links (e.g., wires or wireless links) within a network (e.g., the Internet 105). Network interfaces 210 may be configured to transmit and/or receive data using a variety of different communication protocols, as will be understood by those skilled in the art.

Memory 240 comprises a plurality of storage locations that are addressable by processor 220 for storing software programs and data structures associated with the examples described herein. Processor 220 may comprise necessary elements or logic adapted to execute the software programs and manipulate data structures 245. An operating system 242, portions of which are typically resident in memory 240 and executed by processor 220, functionally organizes device 200 by, inter alia, invoking operations in support of services and/or software processes executing on the device.

These services and/or software processes may comprise an illustrative CoE process/service 244 and a “Content Optimization” process/service 246. Note that while the processes/services are shown in centralized memory 240, as mentioned, it is appreciated that such processes or services can be implemented in a distributed communication environment.

It will be apparent to those skilled in the art of computer engineering that other processor and memory types, including various computer-readable media, may be used to store and execute program instructions pertaining to the techniques described herein. Also, while the description illustrates various processes, it is expressly contemplated that various processes may be embodied as modules configured to operate in accordance with the techniques herein (e.g., according to the functionality of a similar process). Further, while the processes have been shown separately, those skilled in the art will appreciate that processes may be routines or modules within other processes.

Processor 220 can include one or more programmable processors (e.g., microprocessors or microcontrollers, or fixed-logic processors). In the case of a programmable processor, any associated memory 240, may be any type of tangible processor readable memory (e.g., random access, read-only), that is encoded with or stores instructions that can implement program modules (e.g., a module having process 244 encoded thereon). Processor 220 can also include a fixed-logic processing device, such as an application specific integrated circuit (“ASIC”) or a digital signal processor that is configured with firmware comprised of instructions or logic that can cause the processor to perform the functions described herein. Thus, program modules may be encoded in one or more tangible computer readable storage media for execution, such as with fixed logic or programmable logic (e.g., software/computer instructions executed by a processor), and any processor may be a programmable processor, programmable digital logic (e.g., field programmable gate array), or an ASIC that comprises fixed digital logic, or a combination thereof. In general, any process logic may be embodied in a processor or computer readable medium that is encoded with instructions for execution by the processor that, when executed by the processor, are operable to cause the processor to perform the functions described herein.

Illustratively, process 244 and/or process 246 may be performed by hardware, software, and/or firmware, which may contain computer executable instructions executed by processor 220 to perform functions relating to evaluating the intrinsic therapeutic effect of content presented to a user and optimizing content based on the same, as described herein.

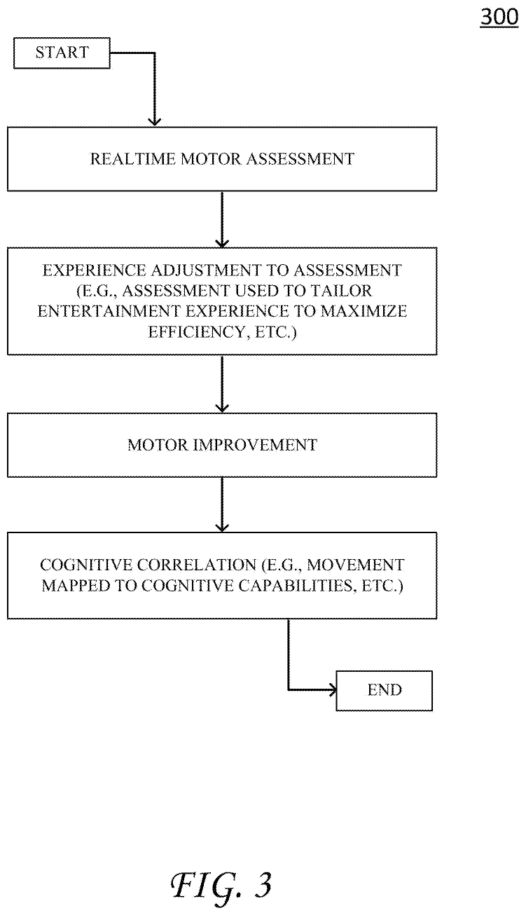

FIG. 3 illustrates a flow diagram 300, showing a high-level Coefficient of Entertainment process. As shown, the CoE process begins with a real-time motor assessment to establish a user/patient baseline.

The second step of the process is a filtering method to introduce change into the contextual experience of the user/patient (description below) in order to assess the impact of the environmental conditions of the context and the user experience.

The third step is a measurement of motor improvement. This is achieved by assessing the current motor score and comparing it with the previous cycle to assess if a change in measurement (“delta”) exists during the session, and also against measurements of previous sessions. This step concerns itself primarily with the assessment of the motor capability as an input to the first stage of the loop.

Example measurements for the motor score are symmetry, skill, and coordination. Symmetry is a measurement of the posture of the body instantaneously and throughout a movement. Skill is the speed of accomplishing a movement. Coordination is the movement of various parts of the body in unison. Measurements are factored according to the difficulty of the movement, and relative to the user's past performance.

The final stage is to assess the cognitive aspect of the context. In all circumstances the user/patient is engaged with entertaining activities that have a cognitive element to them as well as a physical element to them. This measurement is also fed into the initial stage of the loop and is treated in the same way as the motor assessment. This will allow both cognitive and physical deltas to be observed as a context of contextual change and also provide the basis for correlation between motor and cognitive capability whilst eventually providing causation relative to the context.

Example cognitive aspects are the ability to switch attention, remember, calculate mathematics, and navigate visual spatial elements. These cognitive aspects are built into the environment and may be presented as tasks. An example attention switching task includes the alternating between two or more tasks. An example memory task might include the recreation of a previous arrangement of elements in the environment, or an application of facts previously presented. An example mathematical task might include adding or subtracting elements or counting. An example visual spatial task might include the arrangement of elements, or assessment of the arrangement of elements.

Cognitive aspects may be measured by speed and accuracy. Measurements are factored according to the difficulty of the task, and relative to the user's past performance.

While the CoE process shown in FIG. 3 subsequently ends, it is appreciated that this process is an iterative process that may continuously run. Accordingly, the CoE process may continue on to the first step, to perform a real-time motor assessment to establish an updated user/patient baseline of parameters for beginning the next session.

In general, the disclosed comprehensive framework can show and quantify the value that entertainment has in a therapeutic capacity.

The CoE is a metric based on the effectiveness of the entertainment value of the environment. Parameters of various elements in the environment are changed, according to a heuristic framework as well as a stochastic factor. Example parameters might be the color or shape of an element, the audio associated with the session, or the familiarity/popularity of an element.

As changes are introduced, associated measurements of motor and cognitive performance are calculated. Changes to the environmental parameters are attributed to the CoE, reflecting the therapeutic value of entertainment as it pertains to performance.

The disclosed comprehensive framework also provides an evolution of the original calibration to be ongoing and as a part of the experience. This includes the development of an ongoing feedback loop system throughout the experiences, defining the inputs and outputs of the system, defining a study protocol that will be used to test and validate the hypothesis, and removing sources of variance from the dynamic CoE to better quantify it.

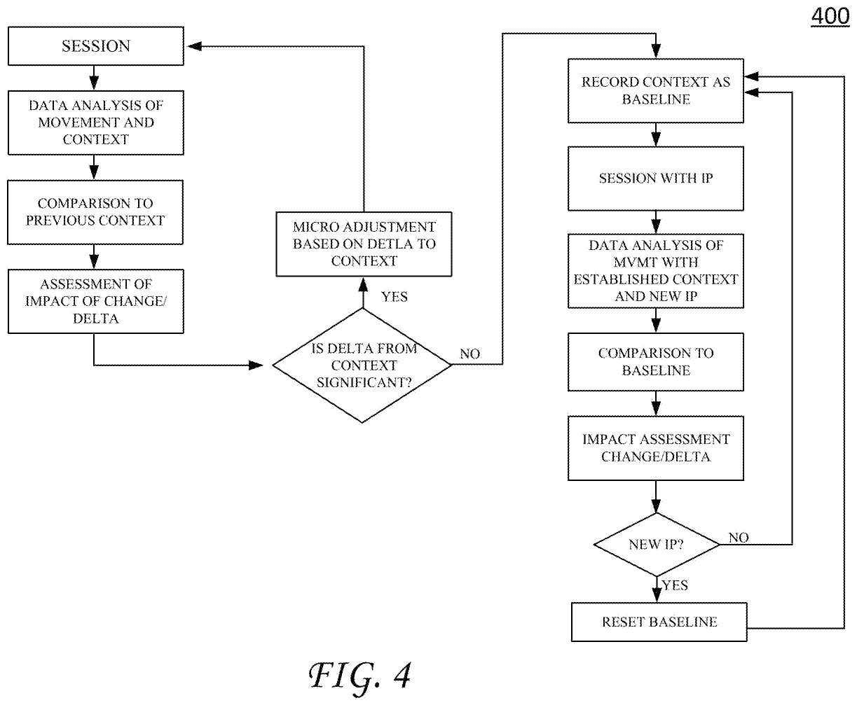

FIG. 4 illustrates a flow diagram 400, showing a high-level filter logic process. As shown, the flowchart represents a cycle, with each cycle the “baseline” of the user is being defined by comparing results from the previous cycle. In one embodiment, the session will consist of sixty cycles per second, a duration and a variable number of activities during that session. Each unique activity within the session is recognized and treated as a separate entity and each 60th of a second that activity is measured against itself in relation to the context, the environment in which the patient is engaged.

During a session it is contemplated that improvement will produce a delta from the beginning of the session until the end of the session. This delta will produce a curve which will be informed and analyzed in conjunction with previous data related to the user/patient. Furthermore, in the second stage of the loop, micro changes will be made to the environment to assess changes in delta that are above a specific threshold. These changes will continue to be applied in the positive or negative until the delta is stabilized. Stable contextual settings will be applied to the user as optimal environmental conditions and tested periodically to ensure statistical validity.

In general, the disclosed comprehensive framework for evaluating the intrinsic therapeutic effect of content presented to a user (e.g., represented as the CoE metric/value) and processes for optimizing content to maximize such therapeutic effect can be described as follows.

There are two general therapeutic focuses of the invention, motor skills and cognitive skills. While these can be focused on separately, they also work inseparably on a physiological level. Enhancing motor skills enhances cognitive skills, and vice versa. The invention seeks to measure and enhance them independently as well as in conjunction.

The invention focuses on occupational therapy movements for enhancing motor skills. Example movements include controlled lifting of a leg while balancing on the other, lifting arms outward and overhead in unison, jogging in place with arms pumping, jumping forward or backward with both feet, etc.

The motor skills are designed to work physical domains, which may be interpreted as body domains. These include individual parts of the body, such as a finger-to-nose, hand, foot, or head.

Domains may also be the grouping of parts into a larger domain, such as the upper arm, entire leg, upper body, lower body, whole body, whole spine, or head and neck. One embodiment of the invention utilizes seventy-two different physical domains.

The invention uses a means of tracking movements of the body. One embodiment of the invention uses a camera to monitor the user, with motion tracking software to find landmarks points on the body. Example points include the crown of the head, base of the neck, sternum, center of the abdomen, center of the pelvis, outer point of the deltoids, elbows, wrists, hips, knees, ankles, etc. The tracking of more landmark points enhances the inventions ability to measure and improve motor skills.

A display prompts the user to perform certain moves. In one embodiment of the invention, the display is a video screen. The display might show an avatar or character performing a variety of moves, and the user is prompted to mirror the moves. Another embodiment might provide a virtual reality object to be manipulated.

The system records the movements. The changes in movement of the landmark points are measured, and provide data for analysis of symmetry, coordination, and speed. The system evaluates performance of moves and may provide the user with feedback. One embodiment provides feedback in the form of a score.

Internally, the system also maintains a score for various aspects of the skill of movements for that particular user. A user demonstrating higher skill in one movement, but lesser skill in another, may prompt the system to emphasize the harder movement in upcoming sessions. Depending on the focus of the session, the system may arrange a variety of harder or easier movements in order to challenge the user, and optimize the therapeutic value of the session.

A variety of motor skills testing systems are available. One embodiment of the invention uses the Bruininks-Oseretsky Test of Motor Proficiency, Second Edition Short Form (“BOT-2”). This is a measure of gross and fine motor skills.

Measuring the improvement of motor skills is evident in the increases score of symmetry, coordination, and speed of performing the movement in subsequent sessions. The increase in these categories also may translate into improvement of other health factors, including ACE-2 activation and cardiovascular health.

The invention also tests cognitive skills through a variety of tasks. Cognitive skills may be divided into a number of domains. Example cognitive domains include attention switching ability, inhibitory control, working memory, visual-spatial ability, and mathematics. Other cognitive domains are also envisioned.

Attention switching is essentially the ability to multi-task. The user is required to perform one task and then required to switch to another task. The user may be prompted to switch back to the first task, required to switch back and forth between two tasks several times, or between three or more tasks.

Inhibitory control is the ability to stop a thought process in order to focus on something new. Inhibitory control is useful to adapt to environments with changing stimuli, where a previous action is no longer appropriate.

Working memory is the ability to hold information temporarily and apply it appropriately. It is useful for decision-making and behavior.

Visual-spatial ability is mentally manipulation of a two-dimensional or three-dimensional object.

A variety of tasks may be presented to the user during a session. One embodiment of the invention has the user interacting in a virtual reality environment. In one embodiment, the user is given a simple mathematical test. The user is asked to signal the answer through a movement, such as touching the correct answer. Another version of an addition task might be placing virtual objects in a container until a designated number is reached.

Another embodiment might include an attention switching challenge involving two separate tasks. The first task might be an association memory game, and another task might be a simple math problem. The user may be given a word and a color, then given a math problem to solve, and then prompted to recall the word given the color as a prompt. Attention switching tasks work multiple cognitive skills at once. This example requires the user to use working memory to remember the word and color, to use inhibitory control to stop focus on the memory task, to switch focus to a math problem, and then to use math skills to solve the math problem. Then, the user must use memory recall to present the word.

Several types of tasks are available, including the following:

An N-back task is a task where a user has to remember whether a stimulus matched a previous stimulus from N steps ago. Means a person needs to keep N number of stimuli in memory. An example might include a spatial position of an object on a field. The object is moved one time, and then a second time. The user is given a blank field and asked to recreate the first position. This is a 2-back task. If the user were asked to recreate the second position, this would be a 1-back task. Generally, the larger the N, the harder the task.

A Spatial Span task is the presentation of an arrangement of objects, that change over the course of multiple iterations. A user is then required to recreate the sequence of arrangements. An example Spatial Span task might be a three-by-three grid of nine squares. One square is shaded in the first iteration, a second square is shaded in a second iteration, and so on. The user is then asked to repeat the sequence of shaded squares.

A Visually Array Change Detection task measures visual working memory. An array of objects in a field are presented. Some of the objects are modified, and the array is displayed again. The user is asked to determine how many, or in what way the objects were modified.

An alternative Change Detection task may also be presented with a distraction element, to measure the working memory.

A Stop Signal task is the differentiation between two stimuli given at random. Two different responses are required for the different stimuli. Response time and accuracy are recorded.

A Go/No-go task tests inhibitory control. Two stimuli are given, with one prompting a positive action, such as pressing a button, and the second prompting a negative action, such as refraining from pressing the button. Response time and accuracy are recorded.

A Modified Stroop test measures selective attention, cognitive flexibility, and processing speed. A typical Stroop test presents two incongruent stimuli, such as a word for a color written in a different color. The word “yellow” might be written in blue color. The user is asked to identify the color of the word, and not the word itself. Response time and accuracy are measured.

A Flanker test measures inhibitory control. Three types of stimuli are provided, congruent, incongruent, and neutral. Stimuli are given at random, and the user is asked to provide appropriate responses.

Additional tests include those provided in the National Institute of Health's NIH Toolbox. This includes over one hundred stand-alone measure for cognition, emotion, motor skills and sensations.

Cognitive domains are measured by performance of the various tasks, for accuracy and response time. Some tasks are objectively more difficult than others and receive greater weight. Over the course of a session, a user may engage in multiple tasks of various difficulty.

The difficulty of individual tasks, and of the collective tasks of an entire session, are dynamically balanced such that a user experiences a high level of success in completing the tasks. One embodiment aims to have the suer perform seventy-five to eighty-five percent of the tasks successfully. Other dynamic balancing regarding the pace of the tasks may be employed to increase or decrease difficulty with respect to limits on response time. If the user is experiencing errors or slow response times, easier tasks may be presented. If the user is experiencing a high rate of success, more difficult tasks may be presented.

Cognitive domains and physical domains are tested simultaneously. The difficulty levels of the cognitive tasks and the physical tasks are coordinated such that one or both are increased or decreased to modify the overall level of difficulty. On the other hand, the difficulty of the cognitive and physical tasks may move inversely, to ease one domain in order to enhance the ability to solve a harder task in the other domain.

Cognitive and physical measurements are maintained in order to assess the correlation between skills, as well as the causation of one domain in enhancing the abilities of the other. The invention utilizes various environmental factors to manipulate the entertainment value of the session. As environmental factors change, the therapeutic entertainment value changes. This change in therapeutic entertainment value is the Coefficient of Entertainment, CoE. The CoE is more than just an aesthetic, enhancing the users experience. It also improves the user's performance, which can be measured by monitoring the user's cognitive and physical domains with respect to adjusting the CoE.

Environmental parameters may be grouped into four categories. These include visual, auditory, familiarity, and pacing.

Visual parameters include lighting, style, color pallet, background movement, and the perceived size of the virtual space.

Some embodiments of the invention are equipped with a means for producing audio. Auditory parameters include volume and pitch. A specific use of volume could be the adjustment of decibel strength in coordination with proximity, to signify a far-off subtle sound form a close-up whisper.

Familiarity refers to the personal preference of, or familiarity to, an environmental characteristic. One example might be a particular song, musical artist, or genre of music. One embodiment of the invention utilizes music familiar in popular culture. Another example might be a particular character, such as a cartoon or superhero.

Pacing refers to the overall feel of the session. Pacing includes a variety of aspects, that create a sense of timing with respect to movements. Example environmental parameters that relate to pacing include, the tempo or rhythm of the audio, the proximity and direction of spatial effects, the emotion of the content, aspects of animation (reserved, lively, emotive tone, aggressive or passive interactivity with the user), dialogue, and visual special effects.

Some environmental parameters may be adjusted by prompts from the user. Other environmental parameters are adjusted stochastically, and continuously measured, to find the optimal set with respect to performance. The CoE related to the optimal set may change over time. Modifications to the environment are constantly implemented in order to recalibrate the CoE.

Some embodiments of the invention utilize gameplay characteristics. These include reward mechanisms to encourage successful performance of the various tasks. Rewards may include audio and visual effects, a point system, and encouragement from other characters within the game. The difficulty and speed of the game, in coordination of reward mechanisms is another aspect of the CoE.

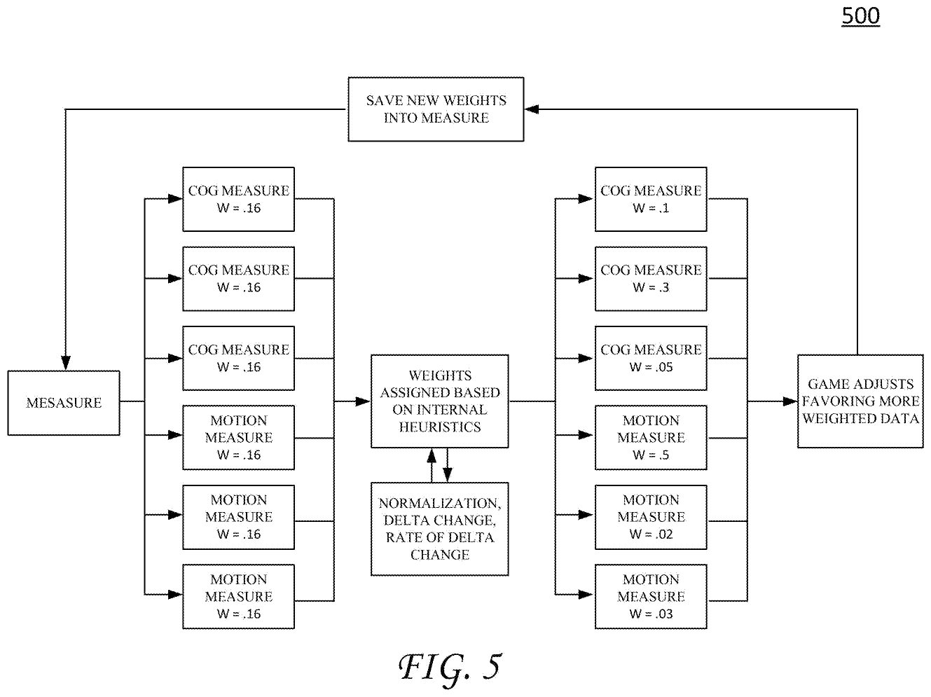

FIG. 5 shows a generalized view of how measurements of cognitive and physical domains generate dynamic suggestions for new cognitive tasks and physical movements. An artificial intelligence process measures performance of all cognitive tasks and physical movements. Example measurements of cognitive tasks are response time and accuracy. Example measurements of physical tasks include symmetry, skill, and coordination.

FIG. 5. illustrates a flow diagram 500, showing an internal weighing adjustment process. As shown, heavily weighted cog and motion measures will be used to adjust game, with minorly weighted ones being ignored. The decision on which to use is based on the rate of change of the measure. If a steep change can be seen, it'll prioritize those measures. If a decline is seen, it'll prioritize the skills that are declining.

Weights are updated and refined with each iteration of new incoming data. Rate of update will be on a game-to-game basis. Longer sessions provide better overall understanding to the weights and avoid stochastic weight updates.

Stochastic updates take in every sample one at a time to update the weights of the model, meaning it may fluctuate heavily. To avoid this for a smoother difficulty curve, the full period should not be used for the weight updates. Only a small subsection should be used, or, the period itself will be assigned different weighting, with newly collected information weighing in heavily in the decision making process with older measurements having less impact. The weighted system can filter out which components are affecting the system most and factor that in when extracting in CoE.

The invention includes real time feedback, and delayed processing feedback. Real time feedback provides the user with an avatar performing the same movements through motion tracking. Delayed processing feedback includes an overall analysis of the session, including the user's cognitive and physical performance, as well as the CoE.

Some embodiments further utilize a human observer. Envisioned observers include parents, teachers, caretakers, researchers, and therapists, in person or remotely. An anticipated benefit of the invention is as an inexpensive method of performing occupational therapy. The invention works under many lighting conditions, spatial constraints, camera types, and body shapes. One potential use could be the assessment of users with autism spectrum disorder. An observer may supplement the invention by also assessing autistic traits during a session with the Social Responsiveness Scale developed by John Constantino.

Another potential use could be assessment of users with Attention Deficit Hyperactivity Disorder. An observer may supplement the invention by also assessing ADHD traits during a session with the Conners Scale developed by C. Keith Conners.

Another role for an observer might be the measurement of everyday executive functions using the Behavior Rating Inventory of Executive Function-2 (“BRIEF-2”).

While there have been shown and described illustrative examples of the comprehensive framework for determining a CoE for VR based content and for optimizing the presentation of content based on the same, it is to be understood that various other adaptations and modifications may be made within the spirit and scope of the embodiments herein. Thus, while the foregoing description has been directed to specific embodiments, it will be apparent that other variations and modifications may be made to the described embodiments, with the attainment of some or all of their advantages. Accordingly, this description is to be taken only by way of example and not to otherwise limit the scope of the embodiments herein.

What is claimed is:

1. A system for presenting physical and cognitive tasks to a user and tracking the user's movements to assess the performance of completing the tasks, the system comprising:
a) a means of displaying visual information to the user,
b) a speaker system for providing audio information to the user,
c) a means of tracking the movements of the user,
d) a means of scoring the movements, and
e) a means of adjusting the visual and audio information to present modified physical and cognitive tasks.

2. The system of claim 1, wherein the means of displaying visual information is one of the following: a television screen, a video projector, a computer monitor, a tablet screen, a mobile phone.

3. The system of claim 1, wherein the means of tracking the movements of the user consists of a video camera providing information to a body analyzer, which finds landmark points on the user's body, and records the absolute and relative positions of the landmark points.

4. A method performed by an information handling system comprising:
a) Displaying visual information prompting a user to perform movements associated with physical and cognitive tasks,
b) Emitting audio information in coordination with the visual information,
c) Tracking the movements of the user,
d) Assessing the movements of the user,
e) Adjusting the visual and audio information to prompt the user to perform movements based on previous assessments.

5. The method of claim 4, wherein the visual information displays an avatar performing the same movements as the user, based on tracking information.

6. The method of claim 4, wherein the assessment of the user's movements is scored based on physical and cognitive aptitude.

7. The method of claim 6, wherein the assessment of the user's movements is scored for physical aptitude based on skill, coordination, and symmetry.

8. The method of claim 6, wherein the assessment of movements is scored for cognitive aptitude based on the time to perform a task, and the accuracy of performing the task.

9. The method of claim 6, wherein the method is executed over a period of time, called a session.

10. The method of claim 9, wherein the assessment of earlier prompted movements provides information to adjust the later prompted movements in the same session.

11. The method of claim 9, wherein the assessment of movements from previous sessions provides information to adjust prompted movements in successive sessions.

12. The method of claim 10, wherein the difficulty of physical and cognitive tasks is adjusted to ensure the probability a 75-85% likelihood of successfully executing the tasks in a session.

13. The method of claim 11, wherein the cumulative difficulty of physical and cognitive tasks in a given session is increased from a previous session.

14. The method of claim 6, wherein the visual and audio information is comprised of entertaining elements.

15. The method of claim 14, wherein the entertaining elements include characters from popular culture.

16. The method of claim 14, wherein the entertaining elements include popular music.

17. The method of claim 14, wherein the assessment of movements is scored in relation to the adjustment of entertaining elements, in order to identify the impact of the entertaining elements.
